(19) United States
(12) Patent Application Publication
(10) Pub. No.: US 2012/0036592 A1
(43) Pub. Date: Feb. 9, 2012

(54) Process for Producing a Hybrid Maize Having Insulated Casing and Individual Shield for Grains, by Means of a Genetic Tool, and a Hybrid Maize Thereof

(76) Inventor: Jean Carlo Landivar Bottega, Leme - SP (BR)

(21) Appl. No.: 13/264,250

(22) PCT Filed: Apr. 16, 2010
(86) PCT No.: PCT/BR2010/000127

§ 371 Date: Oct. 13, 2011

Related U.S. Application Data
(60) Provisional application No. 61/169,961, filed on Apr. 16, 2009.

(51) Int. Cl. A01H 5/00; A01H 5/10; C12N 5/04; A01H 1/02

(52) U.S. Cl. 800/275; 800/320.1; 435/412

(57) ABSTRACT

A process for the generation of hybrid maize whose grains are differentiated and preserved by entirely covering their surfaces, isolating the entire endosperm from the outside and conserving all the nutritious and germinative characteristics from the parentals; and the maize produced by the process.

### FIELD OF THE INVENTION

The present application refers to the generation of hybrid corn.

### BACKGROUND OF THE INVENTION

Nothing similar that has been genetically developed and that is present in any commercial hybrid corn or seed is known today in the prior art. All the other exploits that are normally carried out on corn seeds are chemicals applied by equipment that make this film artificially called coating.

In WO/2008/070939, Bottega describes introduction of anthocyanins in the pericarp of hybrid maize relating. This anthocyanine is of the monomeric type, that is, formed by units easy to extract or separate by water, acids and other common techniques. However, there is still a challenge to find polymeric substances that are fixed to the skin the pericarp of the grain and that act as a protective envelope and that are resistant to averagely harsh environmental conditions for the endosperm of the maize grain.

It was intended then to development a rigorous selection pressure to establish among the known germplasm, which were the healthy grains in harvesting under highly contrasting hydric stress during the cycle. The term “contrasting hydric stress” is defined for the present invention to denominate the combination of extreme drought during the vegetative development of the maize plant and significant rain with permanently high relative humidity during and after the physical maturity up to the grain harvest. This type of climactic condition causes much more specked and rotten than any single condition of long drought or on the contrary any single bout of heavy rain during the entire plant cycle.

This contrast of drought/rain is a very aggressive mechanism for the physiology of the maize plant making it susceptible to grain diseases and loss of quality. This loss of quality can be by physical aggressions directly on the grain surface by the average environmental conditions and insects that break the resistance of the pericarp and subsequently enter fungus, bacteria or virus completing the severe damage to the germ and endosperm. The germ contains the new plant that will flower from the maize seed, and the endosperm contains the nutrients to feed the initial plant before the leaves and roots develop. Accordingly the germ and the endosperm are very rich in nutrients that serve as food for insects or means for bacteria growth, fungus and virus when the pericarp is normally damaged.

The contrasting hydric stress appears very seldom in normal conditions of nature, so artificial conditions must be developed for planting the materials for selection in nurseries.

### SUMMARY OF THE INVENTION

The present description a process for producing a hybrid maize having insulated casing and individual shield for grains, the process comprising the steps of: (a) selecting a germplasm from an existing hybrid lineage with a high content of anthocyanins and high resistance to hydric stress combined with intense irrigation and with additional irrigation at the moment of maturity up to harvest; (b) backcrossing the selected material with the existing hybrid lineage; the germplasm of the existing hybrid lineage used as a female lineage and the germplasm of the selected material used as a male lineage; (c) backcrossing and selecting the backcrossed material with said characteristics for at least five consecutive cycles, and achieving a lineage that is 96.87% similar to the existing lineage and (d) obtaining a uniform maize plant, containing an insulated casing and individual shield for grains, and generating a vegetal material of hybrid maize suitable for industrial and agricultural uses.

### BRIEF DESCRIPTION OF THE DRAWINGS

FIG. 1 shows a cross-section of a regular maize fruit (Zea mays).

FIG. 2 shows a cross-section of the modified maize fruit (Zea mays).

FIG. 3 shows a cross-section of the pericarp of the modified maize.

FIG. 4 shows a front view of the epidermic cells of the pericarp of the modified maize.

FIG. 5 shows the component elements of mesocarp and endocarp of the modified maize.

FIG. 6 shows a front view of the aleuronic cover of the maize.

FIG. 7 shows a cross-sectional view of the aleuronic cover of the maize.

FIGS. 8 and 9 show a cross-sectional view of the parenchymic amylifer of the endosperm of the maize.

### DETAILED DESCRIPTION OF THE INVENTION

In the preferred embodiment of the present invention provides something altogether simple and innovative such as placing an insulated casing on each individual grain by means of a genetic tool. It provides the generation of hybrid maize whose grains are differentiated and preserved by entirely covering their surfaces, isolating the entire endosperm from the outside and conserving all the nutritious and germinative characteristics from the parentals.

The protection is present from the moment in which the seed is planted in breeding fields to form the hybrid corn seed. As soon as it is harvested, dried, classified, packaged, stored, transported, it is distributed to farmers for sowing. Also when the seed is being sown in farmers' fields, and also when the grain is harvested, transported and sold. Also when the grain is dried, stored and delivered to the manufacturers of animal fodder or human food. Additionally, this genetic characteristic is easily transferred to any parental lineage to form part of the new hybrids.

The present description refers to generation of hybrid maize with the uniform inclusion of polymers, immersed inside the cells of the grain's pericarp, which wraps it and provides shielding for its entire surface without using synthetic chemical protectors, but rather genetic means. These polymerized pigments are structurally resistant to harsh rural conditions for mature grain such as: water, temperature, relative humidity, dust (abiotic), insect attack, bacteria or fungus (biotic). It is a total and individual casing for each grain of corn. This shielding is from the seeds to the sowing as well as the harvested grain. The appearance of the grain has a smooth aspect, like shiny and healthy flushed porcelain.

The advantages and characteristics of the modified hybrid maize as described in this specification may be applied in four basis phases:

(1) Transferring the characteristics to the parentals. The characteristics of total and individual shielding for each grain of corn can be transferred from the lineage with the shielding film, as donor in five consecutive retro-breeding and two self-pollinations, to any female lineage that breedingly conforms with the male lineage, to the seeds of the hybrid corn planted by the farmers to harvest the grain. It means that any corn seed and, consequently, the grain harvested by the farmer, will have this shielding characteristic.

(2) Applying on seeds planted by farmers. The seeds having the shielding characteristic, which are used for planting, need not be treated with dye, fungicide or soil insecticide by virtue of the casing shield. The seeds only need systemic insecticide when the plants emerge and the leaves are attacked by insects.

(3) Applying on corn grains harvested in plantations by farmers. The grains harvested from the seeds with the shielding characteristic and planted by the farmers, will include identical shielding to the seeds planted. The quality of the grain will consequently be improved.

The grain is not damaged or present lower quality by external abiotic conditions when the corn grain approaches the time for harvesting or is harvested. These conditions may be: heavy rains, rains with drought intervals, high relative humidity, nights with heavy dew, foggy days, high temperatures during the day, a combination of high temperatures during the day and low temperatures at night, etc. The grain is also not damaged by normal biotic conditions when the corn grain approaches the time for harvesting or is harvested by end-of-cycle sucking or hollowing insects and bacteria or fungus that attack the grain surface.

The grain is also less damaged by cracks and chips from mechanical harvesting processes, transport and movement of the grain along conveyor belts or in elevators.

(4) Applying to stored grains. The grain is less damaged at the moment of air and temperature drying. The grain is also less damaged by insects, and the loss of certain sensitive vitamins and discoloration due to prolonged periods of storage.

Some other useful applications are also possible, such as the pericarp that contains this shielding can be separated when extracted by the industrial corn-processing equipments and its byproducts such as flour, germ, syrup, etc. Once separated, due to the high resistance of this material, it can be used in the manufacture of other materials such as agglomerates, coating for pills, materials with greater resistance than plastics commonly used, high resistance paper, etc.

The characteristics of the corn can also be applied in nutritional effects in human beings by being very stable pigments that might possibly behave differently to the anthocyanins in the digestive system interacting with the absorption of lipids and glucides.

The development of the lineages were carried out in regions where it does not rain during the development of the plants and intense irrigation at the point of maturity and harvesting. The lineages were developed in Canada Larga, Santa Cruz, Bolivia, a very dry, hot and low altitude region where it is not possible to plant and harvest maize. Soon after reaching physiological maturity, irrigation was performed with canon-type high-release equipment, the same that have high penetration of water inside the straw of the cobs of corn to come into contact with the grains and induce lesion of the pericarp and development of micro-organisms that cause poor grains.

A selection was made of all the germplasm that presented anthocyanine in the pericarp originating from some segregating lineages described in WO/2008/070939, namely: A, B, C and D. They were all planted under high hydric stress and combined with intense irrigation and with additional irrigation at the moment of maturity up to harvest.

Plants presenting good health were picked and only the cobs of corn that showed excellent quality of degree, greater than grade 8 of a level of 1 for rotten cobs of corn and 10 for very healthy cobs of corn.

All the cobs of corn after being classified from the most healthy to the least healthy and in this order were left in order in the straw of the same maize that had been picked and passed with knife roll, mixed with wet soil.

This is the best cultivation for fungus, and bacteria and acts as the best inoculum. They were irrigated periodically every 10 days to keep the pressure of the inoculum on the cobs of corn left in the soil.

After 90 days an evaluation was made on how the cobs of corn had withstood the pressure of the conditions of humidity, sun, drought, contact with the earth and the very inocula of the maize plants.

Again an evaluation was carried out to select the largest of 8 and cents of cobs of corn only 1 reached grade 10 of total healthy and 3 cobs of corn achieved grade 9.

Again the cycle starts and grains of grade 10 cob of corn were planted as male and 3 cobs of corn having grade 9 as female to obtain a cross of healthier cobs of corn.

At the time of harvesting, the cobs of corn were harvested with grade 10 that were only 13.

All the degrees of the 13 cobs of corn were planted again, self-pollinating 100% of the plants Fl and again the cobs of corn were picked which presented grade 10 health totaling 27.

All the degrees of the 27 cobs of corn were planted again, self-pollinating 80% of the plants having good characteristics from the agronomic point of view, and at the time of harvesting the cobs of corn were picked that presented grade 10 of health, totaling 37. These 37 cobs of corn were left by order of healthiness again in the wet soil mixed with the maize straw for a further 90 days at the end of which only 16 cobs of corn grade 10 health were selected and with very smooth and bright pericarp. Of the 16 cobs of corn 14 presented pericarp with an intense red color and two low intensity red which at the same time had an appearance of lower quality of degree so they were discarded.

These selected 14 cobs of corn formed the elite germplasm for the quality of degree by a genetic protector. All these cobs of corn, the bright pericarp of the degree of speckling and contrast with the appearance of the pericarp with monomeric anthocyanine which is easily painted during handling.

The selection cycle already exposed was carried out a further 5 times, now in each cycle, placed in the wet soil means with maize straw 10 cobs of corn witnessing the Agricomseeds maize variety AGRI-104 (TRADEMARK) a maize without pericarp protector. The witnesses at the moment of evaluation was always graded lower than 5.

The lineages obtained immediately from all the selection cycles was grade 10 and red color were sent to the laboratory to determine whether they were acting as a protector.

The results obtained confirm that it was no longer monomeric anthocyanine, but rather a very stable pigment of polyphenols with resistance to extraction of supercritical CO₂, water, ethanol, methanol, acetone plus chloroform, acidified methanol, hexane and even hexane plus acetone (analysis No 1). All these chemicals easily extract the anthocyanins, but did not manage to extract the highly stable protective pericarp polyphenols.

Having the lineages with the protective pericarp all with quality grade 10 and which would be the basis of the germplasm, it is necessary to transmit the characteristic of the pericarp for the lineages that is forming a hybrid in the part of the female.

This procedure is very straightforward and has 5 consecutive backcrosses. It is important to use the germplasm source of the protective pericarp as the female; and the female lineage (current lineage) which forms the hybrid to which the protective characteristic is desired to be incorporated, as the male. The resulting seed is used again as the female and the female lineage that forms the hybrid to which the protective characteristic is desired to be incorporated, as the male.

Thus, proceeding until completing 5 backcrosses or 96.87% of similarity with the recurrent lineage. One or two further backcrosses may be required to obtain more similarity with the lineage that is desirable to convert.

Subsequently and whenever necessary the plants obtained should be self-pollinized for two successive cycles to eliminate all the risks of having the desired lineage totally converted and ready for use as an original female to be the hybrid with the protective pericarp.

ANALYSIS 1 OF THE MAIZE

The present analysis used samples of maize originating from genetic material developed by Agricomseeds whose grains are protected by a genetic tool.

The method used was to extract the pericarp to be submitted to a process of tissue disassociation, according to the technique by Jeffrey (Foster, 1950). Other cuts from the fruit were obtained with a handle razor, in a transversal and longitudinal section.

In both cases the samples were submitted to different tints: methylene blue (Stevens, 1916), toluidine blue, safranin (Johansen, 1940) and astra blue plus safranine. To detect starch, Lugol (Johansen, 1940) was used, and for lipidic compounds, Sudán III (Foster, 1949). Subsequently, there samples were mounted between microscope slips, with a drop of water, and observed in an optical microscope and the photograph taken with a camera.

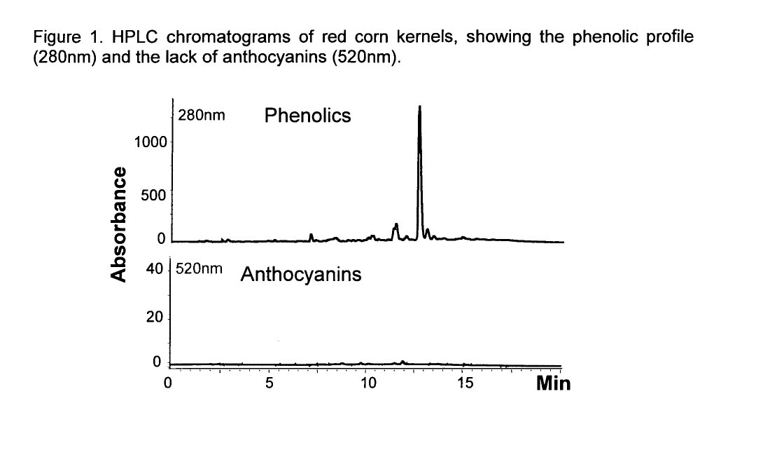

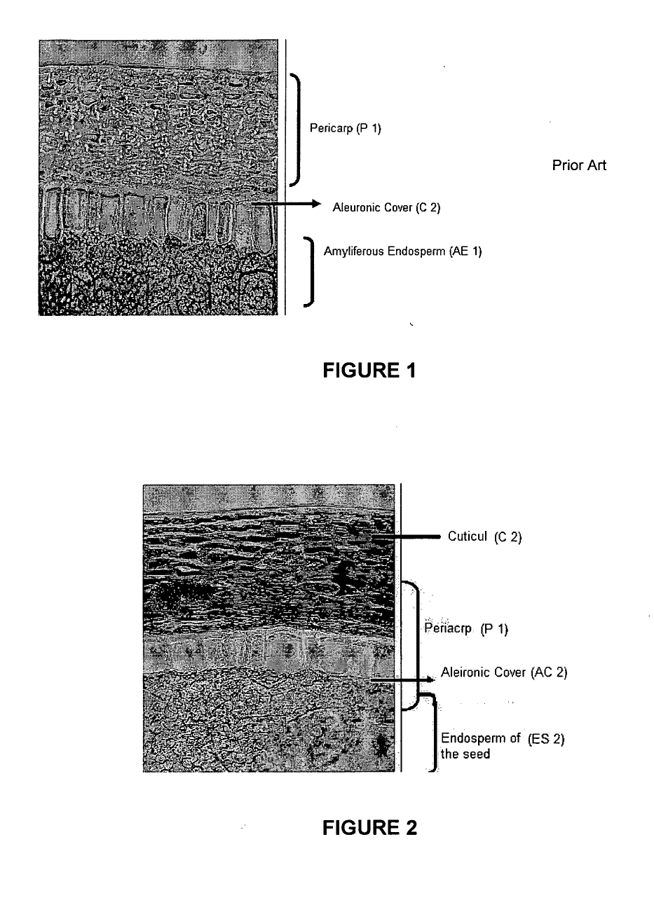

Results: A normal maize (Zea mays L. ) is a cariopsis fruit (FIG. 1) whose pericarp is attached to the seed, which lacks coloring and presents abundant amilyaceous endosperm, with a cotyledon soil where the embryonic axis is inserted. As in the normal maize fruit, the modified maize fruit presents the same structural characteristics, the basic difference being the presence of red pigments (polyphenols) in the cells of all the pericarp, said pigmentation being evidenced in the external aspect of this fruit.

The cross-section of a regular maize fruit (Zea mays) is shown in FIG. 1. The structure includes a pericarp (P 1), a aleuronic cover (AC 1), and an amyliferous endosperm (AE 1).

The cross-section of the modified maize fruit (Zea mays) is shown in FIG. 2. The structure includes a cuticle (C 2), a pericarp (P 2), a aleuronic cover (AC 2), and an endosperm of the seed (ES 2).

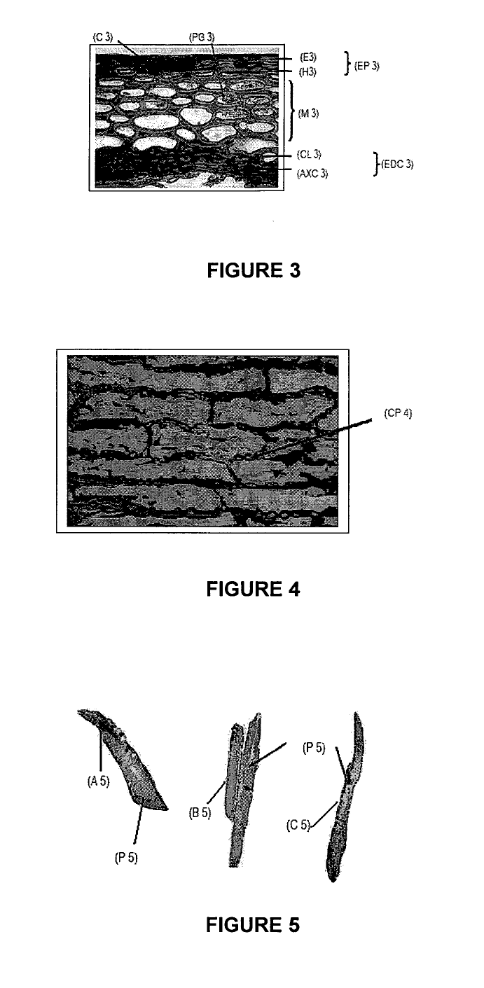

In FIG. 3 it is shown a cross-section of the pericarp of the modified maize. In the pericarp it is possible to distinguish an epicarp (EP 3), a mesocarp (M 3) and an endocarp (EDC 3). The epicarp is shaped by epidermic cell cover and another hypodermis cover; the cells of the epidermis are elongated, rectangular and thickened cellular walls, conspicuous punctuations (see FIG. 4). The pericarp includes a cuticle (C 3), an epidermis (E 3) and an hypodermis (H 3), which are the epicarp (EP 3); then the mesocarp (M 3), and the endocarp (EDC 3), which has transversal cells (CL 3), and axial cells (AXC 3).

As it is seen in FIG. 3, besides a cuticle with a rather thickened cutine coating in the outer cover, due to its chemical nature and structure, it can provide impermeability to water, a mechanical protective function, and resistance to micro-organisms such as insects, fungus and bacteria, as well as to solar radiation and to the entry of contaminating chemical products. The hypodermis is shaped by a cell cover similar to that of the epidermis. In turn, the mesocarp has approximately 5 regular cell covers, with fatty walls and conspicuous punctuations. See FIG. 4, where a front view of the epidermic cells of the pericarp of the maize is shown.

In the endocarp, in turn, two groups of cells are distinguished: the outermost disposed on 3 covers approximately, transversally disposed in relation to the axis of the grain, very thickened cellular walls and conspicuous punctuations; and the innermost comprise a cell cover with axial disposition in relation to the axis of the grain, similar to the previous ones, as it can be seen in see FIG. 5, where a front view of the epidermic cells of the pericarp of the modified maize is shown, where the cellular walls are with conspicuous punctuations. A characteristic of the pericarp is that all its cells contain pigments in the protoplasm, that is, inside the cell.

In FIG. 5 the component elements of mesocarp and endocarp of the modified maize is shown. Where (A 5) are mesocarp cells, and (B 5) and (C 5) are endocarp seed cells. All of them having pigment (P 5).

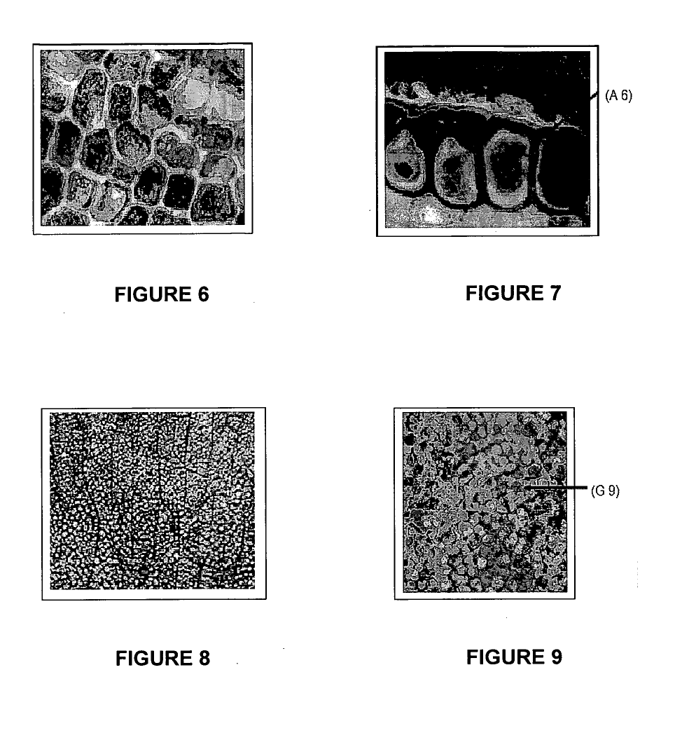

A first cover of parenchymatose cells, with high aleurone content (A 6) is seen in FIGS. 6 and 7.

There are further isodiametric parenchymatose cells that shape the endosperm, as it can be seen in FIGS. 10 and 11.

The structural characteristic that provides protection for the grains of modified hybrid maize described in this specification, with uniform inclusion of polymers immersed inside the pericarp cells of the grain, is the cuticle. The cuticle envelops the grain and gives it total protection for its surface without using synthetic chemical protectors and better still, using genetic tools. This structure provides to the mature grains resistance to harsh conditions such as: rain during harvesting, high or low field temperatures, high relative humidity, dust (abiotic) attack from insects, bacteria or fungus (biotic). In fact, it is a total and individual cover for each grain of maize, from the pre-harvest, harvest and storage. The appearance of the grain becomes very smooth with a bright red and healthy porcelain color.

ANALYSIS 2 OF THE MAIZE

Analysis of phenolics in red corn—Report for AgricomSeeds—by Dr. M. Monica Giusti, Nuryati Pangestu, Department of Food Science and Technology, The Ohio State University 110 Parker Food Science Building, 2015 Fyffe Rd, Columbus, Ohio 43017.

Summary: Pigmented corn samples were received from AgricomSeeds. Samples were analyzed for corn compositional data including anthocyanin content, polymeric anthocyanins, total phenolics, and proanthocyanidins. Neither monomeric anthocyanins nor polymeric anthocyanins were recovered from corn kernels or corn kernels pericarp. However, samples were high in phenolic content, with an average of 64.2 mg phenolics/100 grams dried corn, measured as gallic acid equivalents. In addition, preliminary tests revealed the presence of pro-anthocyanidins in the phenolic extract obtained from whole kernels and from kernel's pericarp. In addition, high content of carotenoids were present.

Pigments responsible for the color of the kernel's epidermal tissue were very stable, resistant to boiling, acid hydrolysis and extraction with common solvents used in the laboratory. They were not monomeric anthocyanins, and further research would be needed to determine their identity.

Comment: The corn samples received show potential for added value due to their high phenolic content, and the presence of pro-anthocyanidins. This new corn cultivar is not recommended as a source of colorant due to the difficulty on extracting the pigments from the tissue. However, the stable coloration could represent a great advantage for food applications.

Procedure: Pigmented corn samples were received from AgricomSeeds. The samples were placed in three different paper bags and treated as 3 replications. Samples were analyzed for corn compositional data including anthocyanin content, polymeric anthocyanins, total phenolics, and proanthocyanidins. Each corn sample was made into a powder using a Waring blendor. Acetone was used for extraction and partitioned with chloroform partition to obtain anthocyanins and other phenolics. The resulting extract was yellow in color and no anthocyanins were found. The cake (insoluble plant material) was yellow in color and powdered skin remained colored (purplish red). Extraction using different solvents (acidified methanol (0.1%), hexane, hexane and acetone (1:1 ratio)) was also performed. Samples were boiled using acidified water (0.1%) to facilitate pigment extraction, and also as a preliminary test for pro-anthocyanidins. Total phenolics were measured on acetone extracts by the Folin-Ciocalteau method for all corn samples.

HPLC Protocol: Samples or red corn kernels were supplied by Agricom Seeds. Samples were extracted with acetone, and partitioned with chloroform, followed by semi-purification by a C-18 cartridge.

A reverse-phase high performance liquid chromatograph (HPLC) system (Shimadzu Corporation, Tokyo, Japan) consisted of LC-20AD prominence liquid chromatograph, a SPD-M20A prominence diode array detector, and SIL-20AC prominence auto sampler at 4° C. LCMS solution Ver3.30 software was used.

Columns and mobile phase: The reversed-phase 3.5 μm Symmetry C18 column (4.6×150 mm, Waters Corp., Milford, Mass., USA) fitted with a 4.6×22 mm Symmetry 2 micro guard column (Waters Corp., Milford, Mass., USA) was used. Solvents and samples were filtered though 0.45 μm nylon membrane filters.

Separation was achieved by using a gradient mobile phase as following: 5% to 25% B, 0-15 min; 25% B, 15-20 min; 25-5% B, 20-25 min. Solvent A was 5% (v/v) formic acid in water and B was 100% acetonitrile. An injection volume of 20 μL with a 0.8 ml/min flow rate was used. Spectral information over the wavelength range of 254-700 nm was collected.

Monomeric and Polymeric Anthocyanin analyses: Corn kernel's pericarp showed a deep red/purple color, typical of anthocyanin pigmentation. A protocol previously used with other corn cultivars (such as Peruvian purple corn) was used to extract and characterize anthocyanins on this new plant material. However, extraction with the typical solvents (acetone, methanol, hot water, Table 1) for anthocyanin analyses resulted on no extraction of monomeric anthocyanins or red color. Extracts were evaluated by Spectrophotometric means and by HPLC analyses. FIG. 1 shows the chromatogram at 520 nm, typical of anthocyanins, and no peaks were detected. There was no monomeric anthocyanin or polymeric anthocyanins detected in the extracts.

Total Phenolics: Each corn sample was made into a powder using a Waring blendor. Acetone was used for extraction and partitioned with chloroform partition to obtain anthocyanins and other phenolics. Total phenolics were measured on acetone extracts as Gallic acid equivalents (GAE) by using the Folin-Ciocalteau method for all corn samples and a Shimadzu dual beam spectrophotometer. Corn kernel samples were high in phenolics, with an average of 64.2 mg GAE/100 grams dried corn kernels. The HPLC analysis (FIG. 1) showed one major peak representing 62% of the total absorbance at 280 nm and 320 nm, indicating the presence of one major phenolic compound. The corn sample is rich in phenolics, with about 3 times the amount reported for mean total phenolic content of conventionally grown and frozen corn (24.7 mg/100 g of fresh weight).

Proanthocyanidins: A preliminary test was performed to evaluate the presence of proanthocyanidins in the corn extracts. Samples were subjected to strong acid/heat treatment. The result was the formation of red color, suggesting the formation of anthocyanidins. Therefore, this preliminary test suggests the presence of proanthocyanidins in the samples. Their identity has not been determined.

Pigment stability: Corn kernels and pericarp tissues were subjected to different solvent treatment, and the coloration on the tissue did not disappear. In addition, tissues were treated with hot water and acidified hot water, and the red/purple coloration remained present. These results indicate that the pigments responsible for the attractive coloration of the corn kernels of the cultivar evaluated were very stable. More research would be needed to elucidate the identification of the pigments.

Spectrophotometric analysis of flavonoids: Anthocyanins, flavonols and phenolic acids were extracted from individual seeds with 1% HCI in 95% ethanol. The extracts were centrifuged twice and their absorption determined spectrophotometrically at 530 nm for anthocyanins, at 350 nm for flavonols and at 280 nm for phenolic acids. The amount of anthocyanins was calculated as cyanidin 3-glucoside equivalents (molar extinction coefficient (ε) 26900 L nVmoh¹. M. W. 484.82), flavonols content as quercetin 3-glucoside equivalents (ε 21877 L nr¹mol−¹, M. W. 464.38) and the amount of phenolics as ferulic acid equivalents (ε 14700 L m−¹mol−¹, M. W. 194.18). Mean values represent seven independent replicates ±S.D. Phlobaphenes were extracted from individual seeds with 1 volume of concentrated HCI and 4 volumes of dimethylsulfoxide (DMSO) sequentially with vigorous vortexing after each addition, essentially as described by Das et al. (28). Extracts were then centrifuged and cleared supernatants were diluted with methanol (20% final concentration). Phlobaphenes concentration was expressed as absorbance value at their Amax (510 nm) per g of dry weight ±S.D.

TABLE 1
Qualitative observations with different extraction solvents:

| | Color of | Color of pellet | Other |
|---|---|---|---|
| Solvent | solution | (cake) | observation |
| 70% acetone/ | yellow | The insoluble | The powdered |
| chloroform | | plant material was | skin remained |
| partition | | yellow. | colored (purplish |
| | | | red). |
| Acidified methanol | yellow | The insoluble | The powdered |
| (0.1%) | | plant material was | skin remained |
| | | yellow. | colored (purplish |
| | | | red). |
| Hexane | yellow | The insoluble | The powdered |
| | | plant material was | skin remained |
| | | yellow. | colored (purplish |
| | | | red). |
| Hexane and | yellow | The insoluble | The powdered |
| acetone (1:1 ratio) | | plant material was | skin remained |
| | | yellow. | colored (purplish |
| | | | red). |

1. A process for producing a hybrid maize having insulated casing and individual shield for grains , the process comprising the steps of:
(a) selecting a germplasm from an existing hybrid lineage with a high content of anthocyanins and high resistance to hydric stress combined with intense irrigation and with additional irrigation at the moment of maturity up to harvest;
(b) backcrossing the selected material with the existing hybrid lineage; the germplasm of the existing hybrid lineage used as a female lineage and the germplasm of the selected material used as a male lineage;
(c) backcrossing and selecting the backcrossed material with said characteristics for at least five consecutive cycles, and achieving a lineage that is 96.87% similar to the existing lineage and
(d) obtaining a uniform maize plant, containing an insulated casing and individual shield for grains, and generating a vegetal material of hybrid maize suitable for industrial and agricultural uses.

2. A process for producing a hybrid maize having insulated casing and individual shield for grains , the process comprising the steps of:
(a) selecting a germplasm from an existing hybrid lineage with a high content of anthocyanins
(b) planting the germplasm and selecting the obtained cob material from maize plants, with high resistance to hydric stress combined with intense irrigation and with additional irrigation at the moment of maturity up to harvest;
(c) backcrossing the selected material with the existing hybrid lineage; the germplasm of the existing hybrid lineage used as a female lineage and the germplasm of the selected material used as a male lineage;
(d) classifying the selected cob material from the most healthy to the least healthy on a scale ranging from 1 for rotten cobs of corn and 10 for very healthy cobs of corn;
(e) selecting the cob material greater than grade 8 within the scale of 1 to 10;
(f) storing the selected cob material in the straw of its own maize plant, the straw previously disintegrated through a knife roll and mixed with wet soil, promoting inoculation and cultivation for existing fungus and bacteria present in the straw;
(g) irrigating the stored material periodically;
(h) selecting the stored cob material, with high resistance to humidity, sun, drought, contact with the earth and the very inocula of the maize plants, within the scale of 1 to 10;
(i) planting grains of cob of maize grade 10 as male and grains of cob of maize grade 9 as female;
(j) selecting and harvesting the planted cob material grade 10, within the scale of 1 to 10;
(k) promoting self-pollinating of 100% of the selected obtained plants F1;
(I) selecting and harvesting the planted cob material grade 10, within the scale of 1 to 10;
(m)planting grains of cob of maize grade 10 (as male and grains of cob of maize grade 9 as female) promoting self-pollinating of 80% of the selected obtained plants of the plants having good characteristics from the agronomic point of view;
(n) selecting and harvesting the planted cob material grade 10, within the scale of 1 to 10;
(o) storing the selected cob material in the straw of its own maize plant, the straw previously disintegrated through a knife roll and mixed with wet soil, promoting inoculation and cultivation for existing fungus and bacteria present in the straw;
(p) irrigating the stored material periodically;
(q) selecting the stored cob material grade 10 and with very smooth and bright pericarp with an intense red color, the material carrying the desired germplasm for the quality of degree by a genetic protector lineage that is 96.87% similar to the existing lineage, and
(r) obtaining a uniform maize plant, containing an insulated casing and individual shield for grains, and generating a vegetal material of hybrid maize suitable for industrial and agricultural uses.

3. The process for producing a hybrid maize having insulated casing and individual shield for grains according to claim 1, wherein the uniform maize plant plants obtained in step (d) is further self-pollinated for two successive cycles to eliminate all the risks of having the desired lineage totally converted and ready for use as an original female to be the hybrid with the protective pericarp.

4. The process for producing a hybrid maize having insulated casing and individual shield for grains according to claim 2, wherein the uniform maize plant plants obtained in step (r) is further self-pollinated for two successive cycles to eliminate all the risks of having the desired lineage totally converted and ready for use as an original female to be the hybrid with the protective pericarp.

5. The process for producing a hybrid maize having insulated casing and individual shield for grains according to claim 2, wherein in step (h) the selection of the stored cob material, with high resistance to humidity, sun, drought, contact with the earth and the very inocula of the maize plants, within the scale of 1 to 10; is performed after a period of ninety days.

6. The process for producing a hybrid maize having insulated casing and individual shield for grains according to claim 1, wherein the grains of the hybrid maize have the physiological and morphological characteristics of having an insulated casing on each individual grain entirely covering the grain surfaces and isolating the entire endosperm from the outside and conserving all the nutritious and germinative characteristics from the parentals.

7. The process for producing a hybrid maize having insulated casing and individual shield for grains according to claim 1, wherein the grains of the hybrid maize have the physiological and morphological characteristics of having a uniform inclusion of polymers, immersed inside the cells of the grain's pericarp, the pericarp wraping and providing shielding for the entire surface of the grain.

8. The process for producing a hybrid maize having insulated casing and individual shield for grains according to claim 1, wherein the hybrid maize plant has the physiological and morphological characteristics of: (1) stress tolerance due to lack of water resistance to unsuitable amount of water, (2) resistance to inadequate temperature and relative humidity (3) resistance to dust, (5) resistance to insect, bacteria or fungus diseases.

9. The process for producing a hybrid maize having insulated casing and individual shield for grains according to claim 2, wherein the pericarp having the shield of the hybrid maize plant has the physiological and morphological characteristics of being able to be separated when extracted by the industrial corn-processing equipments and the byproducts, and the separated pericarp able to be used in the manufacture of materials of greater resistance.

10. The process for producing a hybrid maize having insulated casing and individual shield for grains according to claim 2, wherein the grains of the hybrid maize have the physiological and morphological characteristics of having an insulated casing on each individual grain entirely covering the grain surfaces and isolating the entire endosperm from the outside and conserving all the nutritious and germinative characteristics from the parentals.

11. The process for producing a hybrid maize having insulated casing and individual shield for grains according to claim 2, wherein the grains of the hybrid maize have the physiological and morphological characteristics of having a uniform inclusion of polymers, immersed inside the cells of the grain's pericarp, the pericarp wraping and providing shielding for the entire surface of the grain.

12. The process for producing a hybrid maize having insulated casing and individual shield for grains according to claim 2, wherein the hybrid maize plant has the physiological and morphological characteristics of: (1) stress tolerance due to lack of water resistance to unsuitable amount of water, (2) resistance to inadequate temperature and relative humidity (3) resistance to dust, (5) resistance to insect, bacteria or fungus diseases.

13. The process for producing a hybrid maize having insulated casing and individual shield for grains according to claim 2, wherein the pericarp having the shield of the hybrid maize plant has the physiological and morphological characteristics of being able to be separated when extracted by the industrial corn-processing equipments and the byproducts, and the separated pericarp able to be used in the manufacture of materials of greater resistance.

14. A seed of the hybrid maize produced according to the process of claim 1.

15. A seed of the hybrid maize produced according to the process of claim 2.

16. A hybrid maize plant, or a part thereof, produced by growing the seed according to claim 14.

17. A hybrid maize plant, or a part thereof, produced by growing the seed according to claim 15.

18. Pollen of the hybrid maize plant according to claim 16.

19. Pollen of the hybrid maize plant according to claim 17.

20. An ovule or ovules of the hybrid maize plant according to claim 16.

21. An ovule or ovules of the hybrid maize plant according to claim 17.

22. A tissue culture of regenerable cells produced from the hybrid maize plant according to claim 16.

23. A tissue culture of regenerable cells produced from the hybrid maize plant according to claim 17.

24. Protoplasts or callus produced from the tissue culture according to claim 22.

25. Protoplasts or callus produced from the tissue culture according to claim 23.

26. The tissue culture according to claim 22, wherein the regenerable cells of the tissue culture are produced from protoplasts or from tissue of a plant part selected from the group consisting of: immature embryo, embryo, meristematic cells, immature tassels, microspores, pollen, root, root tip, anther, silk, leaf, flower, kernel, ear, cob, husk and stalk.

27. The tissue culture according to claim 23, wherein the regenerable cells of the tissue culture are produced from protoplasts or from tissue of a plant part selected from the group consisting of: immature embryo, embryo, meristematic cells, immature tassels, microspores, pollen, root, root tip, anther, silk, leaf, flower, kernel, ear, cob, husk and stalk.
